(19) United States
(12) Patent Application Publication
(10) Pub. No.: US 2008/0015639 A1
(43) Pub. Date: Jan. 17, 2008

(54) Anchorless non-invasive force dissipation system for orthopedic instrumentation

(76) Inventors: Todd M. Bjork, River Falls, WI (US); Dan McPhillips, Ham Lake, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/655,730

(22) Filed: Jan. 19, 2007

Related U.S. Application Data
(60) Provisional application No. 60/760,144, filed on Jan. 19, 2006.

(51) Int. Cl. A61F 5/00; A61B 17/58

(52) U.S. Cl. 606/240; 606/237; 606/90

(57) ABSTRACT

An anchorless non-invasive force dissipation device for orthopedic instrumentation including a base having a patient contacting surface, the patient contacting surface including a surface area adapted for external placement on a patient's body, and an instrument alignment mechanism operably connected to and selectively positionable relative to the base, the instrument alignment mechanism adapted to interface with at least one orthopedic instrument, such that forces applied by the orthopedic instrument are dissipated across the surface area of the base with the device being unanchored externally of the patient.

### RELATED APPLICATION

The present application claims the benefit of U.S. Provisional Application No. 60/760,144, filed Jan. 19, 2006, which is incorporated herein in its entirety by reference.

### FIELD OF THE INVENTION

This invention relates to methods and devices for dissipating applied forces and maintaining the alignment of orthopedic instrumentation. More particularly, the present invention relates to method and devices for dissipating applied forces and maintaining the alignment of orthopedic instrumentation that are non-invasive and do not need to be anchored external to the patient.

### BACKGROUND OF THE INVENTION

In many surgical procedures, especially in orthopedics, force is necessarily applied to instruments that are placed within a patient's body in order for those instruments to perform their intended function. Mechanical force is applied to the instrument while it is positioned at the surgical site deep inside the patient's body. When this force is applied, there is potential danger for harming the patient. For instance in orthopedic procedures, bone filler material is often pounded or tamped into the desired interior body region of the patient. This pounding and/or tamping imparts force on to the patient which is necessary to achieve the intended function at the targeted surgical site, but which may cause collateral damage to other body structures or tissues.

Devices for alignment and force dissipation for orthopedic instrumentation are typically either invasive or non-invasive. Invasive devices are typically pinned, screwed or otherwise secured to the patient, such as to a bone in the patient. Examples of such invasive devices are shown in U.S. Pat. Nos. 6,893,447 and 6,921,404. In addition to the trauma caused by anchoring the invasive device to the patient, such invasive devices can only be used when the patient is under general anesthesia.

Conventional non-invasive devices used for alignment and force dissipation typically comprise an external frame construct that is mounted and locked onto the surgical table, such as shown in U.S. Pat. Nos. 4,355,631, 4,718,151, and 5,242,240. While these kinds of frames stabilize the instruments and dissipate impact forces by redirecting them to the table itself, they can be cumbersome to set up and use. Further, because the frame is locked to the surgical table, and the instruments linked to the frame are positioned relatively deeply within the patient's body, any motion of the patient during a procedure may potentially pose a risk of injury to the patient. The alignment and stability of the instruments may also be lost with such motion. Because of this potential risk of injury and loss of instrument alignment, general anesthesia is the recommended anesthesia treatment option for use with conventional table mounted systems.

General anesthesia renders the patient immobile, thus eliminating the sensation of pain by the patient and diminishing the risk of patient movement during the procedure. However, general anesthesia does have some potential drawbacks such as possible postoperative nausea, vomiting and somnolence. Further, because general anesthesia affects the central nervous system and depresses the patient's vital signs, the recovery time is longer than other anesthesia options. The potential for adverse side effects from the use of general anesthesia causes many surgeons to consider other anesthesia options when possible. In the case of devices for alignment and force dissipation for orthopedic instrumentation, such options are generally not available with current invasive or non-invasive alignment and force dissipation devices.

Therefore, there is a need for an easy to use, safe and effective non-invasive device which dissipates the force applied in surgical procedures without introducing added risks if the patient moves during the procedure, and without overloading other structures or tissues of the patient's body. Such a system will reduce the risk of potential injury to the patient and will broaden the available anesthesia options.

### SUMMARY OF THE INVENTION

The device of the present invention includes an anchorless non-invasive force dissipation device for orthopedic instrumentation that may include a base having a patient contacting surface. The patient contacting surface may include a surface area adapted for external placement on a patient's body. An instrument alignment mechanism may be operably connected to and selectively positionable relative to the base. The instrument alignment mechanism may be adapted to interface with at least one orthopedic instrument, such that forces applied by the orthopedic instrument are dissipated across the surface area of the base with the device being unanchored externally of the patient.

Many systems exist to guide drills, cutters, tamps and other surgical instruments into interior body regions. The present invention is an improvement over such devices because it not only serves to guide and stabilize the surgical instruments, it also dissipates the mechanical force applied to such instruments. Dissipation of applied impact and pressure forces helps to protect the local tissues. The placement of the device directly onto the patient permits freedom of patient movement. If a procedure is conducted with sedation and monitored anesthesia care (MAC) as opposed to general anesthesia, the patient is capable of movement. As discussed above, patient movement can become a significant consideration in surgical procedures where a guidance system is locked onto the operating table.

In use, the device of the present invention may be positioned on the patient once the desired treatment location and instrument insertion trajectory has been established. The base of the device stabilizes the instruments by positioning an intermediate stop against the skin surface and dissipates the force imparted by instruments by distributing it over a relatively large area in order to minimize the contact force at any one location. A working cannula may be positioned on the base to guide the instruments to the desired interior body location and maintain both depth control and the desired trajectory for instrument insertion.

Once the working cannula has been positioned at the interior bony surgical site, the trajectory of the instruments is maintained by virtue of the cannula's placement through the soft tissues located between the patient's skin and the bone. If the patient moves or twists, the surgeon simply releases his/her hands from the instruments momentarily, but the working trajectory will be preserved and the procedure can resume as soon as the patient's motion has stopped, thus alleviating the potential risk of injury to the patient associated with patient movement and conventional table mounted systems.

In one embodiment of the present invention, the base of the device is comprised of a polyetherimide and comprises a generally flat patient contacting surface. A working cannula may be freely positionable on the base using a ball joint.

In another embodiment of the present invention the patient contacting surface of the base may be curved to fit the contours of a patient's body. The patient contacting surface may further include an adhesive, foam or other coating to assist in positioning the base to the patient.

In yet another embodiment the device includes a conformable pad, separate from, but used in conjunction with the base plate to conform to the particular contours of varying patient's bodies.

In another embodiment of the present invention, the base may have a surface area in the range of about 4 square inches to 20 square inches, a thickness in the range of about one-quarter (¼) inch to about 1 and one-half (1½) inches, Shore D hardness in a range of about 60 to 90, and may withstand applied forces of up to about 20,000 psi.

In an embodiment of the present invention the instrument alignment mechanism is centrally mounted on the base. Because the instrument alignment mechanism is centrally mounted on the base the applied force from the instruments is colinear with the point of action and thus there is little bending force applied to the instruments.

In yet another embodiment of the present invention the force dissipation device may include adjustable heads to adjust the length of the sheath. One or more of the adjustable heads may include markings to visually gauge the depth of the working cannula within the surgical site.

In another embodiment, removable block portions may be used to adjust the length of the sheath and thus the depth stop of the device such that the surgeon can vary the depth that the working cannula is inserted into the surgical site.

### BRIEF DESCRIPTION OF THE FIGURES

FIG. 1 is a top plan view of an embodiment of the force dissipation device of the present invention.

FIG. 2 is a side elevational view of an embodiment of the force dissipation device of the present invention.

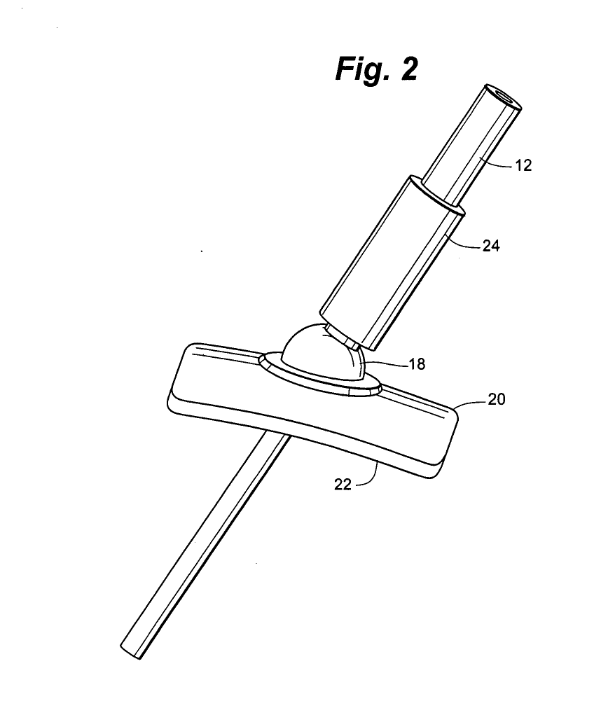

FIG. 3 is an alternative embodiment of the force dissipation device of the present invention.

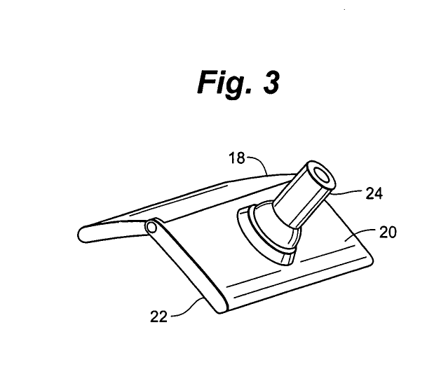

FIG. 4 is a side elevational view of another alternative embodiment of the force dissipation device of the present invention.

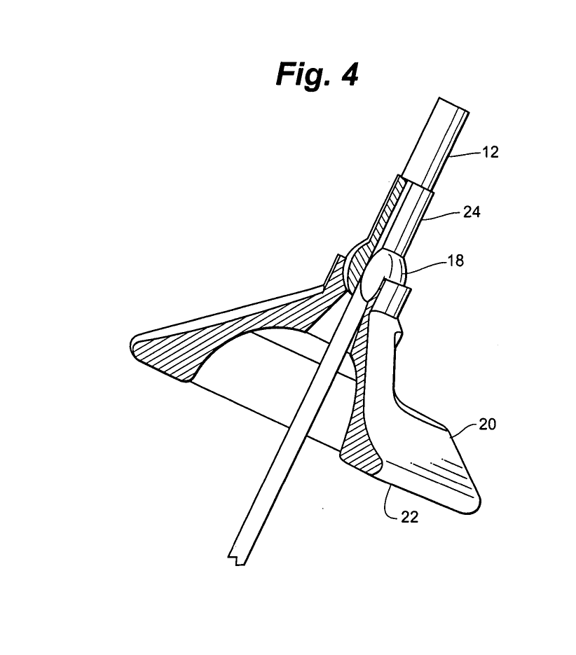

FIG. 5 depicts a preferred embodiment of the force dissipation device of the present invention in use.

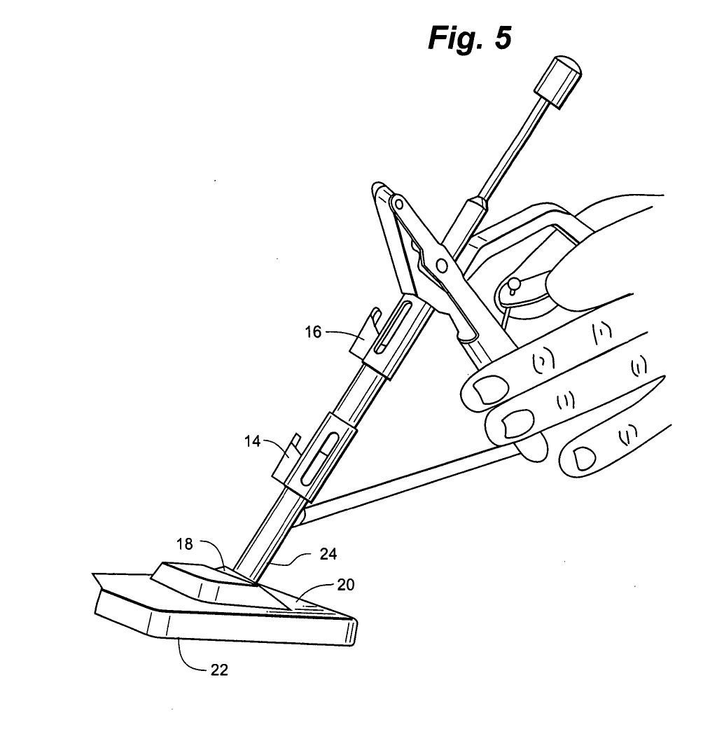

FIG. 6 depicts a top view of an alternate embodiment of force dissipation device of the present invention.

### DETAILED DESCRIPTION OF THE INVENTION

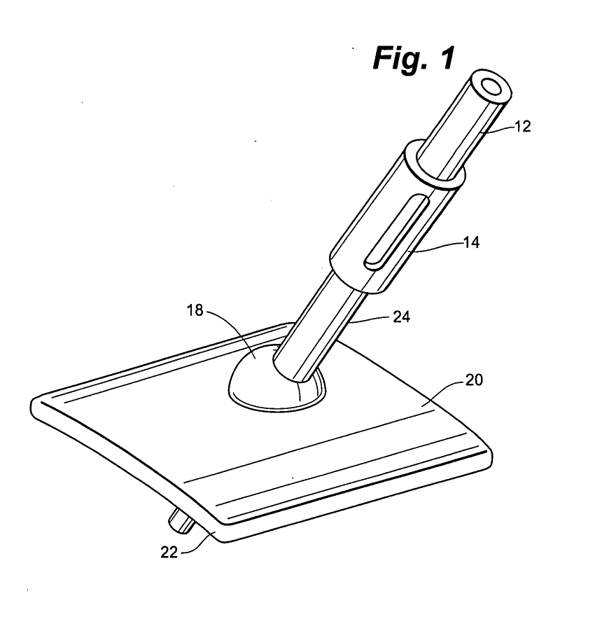

The device of the present invention maintains the desired insertion trajectory of medical instruments and dissipates the force imparted by these same medical instruments by dispersing it over a relatively large area at the patient's skin surface. As is shown in FIG. 1, the device 10 comprises a base 20 and an interchangeable sheath component 24 attached to a freely positionable ball joint 18. The sheath 24 may be attached to any mechanism which is freely positionable in infinite degrees of freedom. The base 20 may be constructed to conform to the contours of the patient's body. The base 20 may be constructed of plastics, polymers, Kevlar® or any other suitable medical grade material. In a preferred embodiment, the base 20 is constructed of a polyetherimide, such as Ultem® plastic.

The base 20 is positioned directly on the patient, providing safety benefits over conventional systems. Because of the potential side effects of general anesthesia and other considerations, many orthopedic procedures are performed under monitored anesthesia care or “MAC.” MAC anesthesia includes the use of local anesthesia such that the patient is numb at the surgical site. The patient is usually also given intravenous medication to calm and relax them during the procedure. The anesthetist or anesthesiologist then monitors the patient during the procedure.

Patients are awake during MAC anesthesia, and thus the patients may inadvertently move during surgery. If, as in conventionally mounted systems, the working cannula 12 is secured to the operating table, relative movement between the patient and the cannula 12 may result in a loss of instrument alignment and stability. Mounting the base 20 and the sheath 24 directly to the surface of the patient offers an important safety benefit over these conventional systems. If a patient moves during a procedure, the surgeon can simply let go of the instruments until such movement stops, all the while the instrument alignment and stability remains intact.

Similarly, relative movement under impact or pressure loads can occur along the axis of the working cannula 12 in a system which is not mounted to the patient. Tamping which occurs through the working cannula 12, for example, may push the patient's bone away from its engagement point at the distal end of the working cannula 12. When the tamping force or pressure is released and the patient's bone returns or springs back to its starting position, the relative position of the working cannula 12 against the bone may be different, and may offer a risk of entrapping tissue between the end of the working cannula 12 and the bone. The patient-mounted base 20 and working cannula 12 of the present invention minimize the opportunity for significant relative motion to occur between the patient and any of the surgical instrumentation.

The base 20 includes a patient contacting surface 22. The patient contacting surface 22 may include an adhesive to aid in the positioning and stability of the base 20. The patient contacting surface 22 may further include foam or other suitable cushioning material. When mechanical forces are imparted onto the medical instruments passing through the working cannula 12, those forces are dissipated against the depth stop of the working cannula 12 and the sheath 24 and subsequently across the surface area of the base 20. Thus, the impact forces that reach the interior body regions are partially controlled and are targeted to the surgical site. In the absence of the force dissipation, the mechanical forces imparted by the medical instruments could cause severe damage to tissues and structures apart from the surgical site.

A surgical access portal or working cannula 12 is positioned on the base 20 to guide the placement of the instruments into the desired interior body region. The working cannula 12 controls the depth and insertion trajectory for the instruments introduced within and through the cannula 12 into the surgical site. The working cannula 12 may be slidably received through the sheath 24 of the device with its freely positionable ball joint 18. A locking mechanism may be employed to lock the working cannula 12 into a desired position relative to the base (or interior body region). In an embodiment of the present invention, the locking mechanism may comprise a split channel and collar system such that the access portal includes channels aligned parallel or slightly toward each other and a collar movable in a longitudinal direction such that the channels are moved apart thus locking the working cannula 12 in place. Conversely, the channels can be brought together releasing the working cannula 12.

The working cannula 12 may include depth gauges, such as markings to indicate how deep the working cannula 12 is placed into the patient's interior body region. The device 10 may further include a mechanism to adjust the length of the sheath 24. Such a mechanism may include interchangeable blocks of various heights that may be placed on the sheath 24 that allow the user to vary the length of the sheath 24. The sheath 24 may also be telescoping to vary its length. In another embodiment the device 10 may include adjustable heads 14 and 16 to vary the length of the sheath 24. The adjustable heads may include a spring loaded push button to slidably adjust the length of the sheath 24. By adjusting the length of the sheath 24, which acts as a depth stop for the cannula, the depth that the cannula is inserted into the surgical site may be varied.

The preferred embodiment of the present invention will be described as it is used in the treatment of a vertebral body defect such as a compression fracture. The device 10 includes a base 20, which may be constructed of any suitable medical grade material, such as plastic or Kevlar®. Prior to placement of the device 10, the surgical site is identified by placement of a conventional guide pin into the vertebral defect. The safe and proper position of this pin is selected using fluoroscopic guidance to permit visualization by the surgeon.

Following placement of the pin, a cannulated dilating device is placed. The cannulation of the dilator closely fits over the pin diameter. The body of the dilator serves to create a larger access path through the patient's tissue by gently deflecting tissues in its path. Placement of the dilator can, in one embodiment, aid in selecting the appropriate length and depth of the interchangeable sheath 24. This sheath 24 selection can be accomplished by observing depth markings on the body of the dilator at the point where the dilator crosses the surface of the patient's skin.

The chosen sheath 24 with its freely positionable ball joint 18 may then be quickly assembled to the base 20. The sheath 24 is guided over the dilator and the base 20 is carefully positioned on the patient's skin at the resulting location. The cannula 12 is then placed over the dilator and through the sheath 24, and advanced to its final docking position in bone. The base 20 may be secured, if needed, to the patient's skin using foam, tape or similar adhesive and/or fixation means.

Once the base 20 is secured, the dilator and guide pin may be removed such that the working cannula 12 is positioned for the introduction and guidance of all subsequent instruments needed to complete the procedure. The working cannula 12 provides a safe, repeatable trajectory for the passage of all subsequent instruments. In an embodiment of the present invention, the instrument alignment mechanism may include the sheath 24, its freely positionable ball joint 18 and the working cannula 12. In procedures where the instruments used must be operated with some degree of force, the working cannula 12 and base 20 together serve to transfer a portion of the applied force to the outer surface of the patient's body and to dissipate that force over a broader surface area, minimizing the contact loading against the patient's body and body tissues. The localized contact loading is minimized because the base 20 provides a greater surface area than the end of the instruments themselves, thus decreasing the pounds of force transferred to the surface at any given square inch.

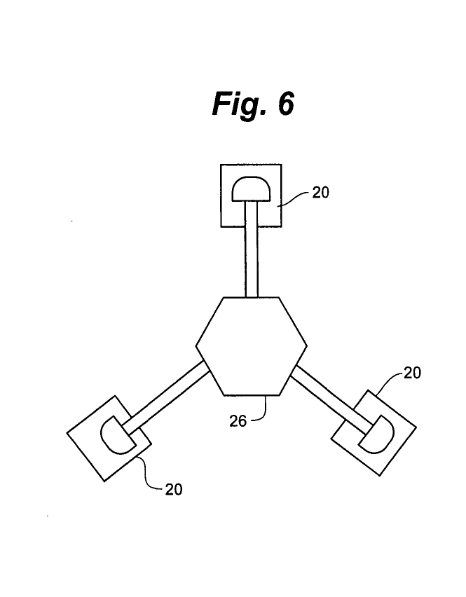

As shown in FIG. 6, in an alternate embodiment of the present invention, the device may include more than one base 20, in an outrigger configuration. Preferably, this outrigger configuration may include at least 3 bases 20 placed on the patient's body. Each base may include an instrument alignment mechanism. The instrument alignment mechanism of each base is operably connected to each of the other instrument alignment mechanisms at least one juncture 26 outside the perimeter of the bases.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

1. An anchorless non-invasive force dissipation device for orthopedic instrumentation comprising:
a base having a patient contacting surface, the patient contacting surface including a surface area adapted for external placement on a patient's body such that applied forces are dissipated across the surface area, and
an instrument alignment mechanism operably connected to the base and selectively positionable in at least 2 degrees of freedom relative to the base.

2. The device of claim 1, wherein the device is unanchored externally of the patient.

3. The device of claim 1, wherein the instrument alignment mechanism includes a sheath, a structure defined by a lumen, having a proximal end and a distal end, the distal end including a depth stop, the sheath being positionable in at least 2 degrees of freedom on the base and a working cannula slidably received through the lumen.

4. The device of claim 3, wherein the length of the depth stop is adjustable.

5. The device of claim 4, wherein the depth stop includes adjustable heads to adjust the length of the depth stop.

6. The device of claim 1, wherein the instrument alignment mechanism is positioned on the base using a ball joint.

7. The device of claim 1, wherein the dissipated applied forces may be up to 20,000 psi.

8. The device of claim 1, wherein a thickness of the base is in the range of one-quarter inch to one and one-half inch.

9. The device of claim 1, wherein the surface area of the base is in the range of four square inches to 20 square inches.

10. The device of claim 1 wherein the instrument alignment mechanism further includes means for locking the at least two degrees of freedom once a desired position is achieved.

11. A medical device for dissipating force comprising:
a base having a patient contacting surface, the patient contacting surface including a surface area adapted for placement on a patient's body such that applied forces are dissipated across the surface area,
a sheath having a proximal end and a distal end, the distal end including a depth stop, the sheath being positionable in at least 2 degrees of freedom relative to the base, and
a working cannula slidably received through the depth stop.

12. The device of claim 11, wherein the length of the depth stop is adjustable.

13. The device of claim 12, wherein the depth stop includes adjustable heads to adjust the length of the depth stop.

14. The device of claim 11, wherein the depth stop is positioned on the base using a ball joint.

15. A method for dissipating force applied to a patient comprising the steps of:
placing a base having a patient contact surface area onto the patient such that applied forces are dissipated across the surface area,
positioning a sheath relative to the base, and
sliding a working cannula through the sheath and into the surgical site.

16. The method of claim 15 further including the step of locking the sheath and the working cannula into position establishing a repeatable trajectory for the insertion of the surgical instruments.

17. A method for dissipating force applied to a patient comprising the steps of:
placing a base having a patient contacting surface area on a patient,
positioning an instrument alignment mechanism relative to the base,
positioning at least one orthopedic instrument to interface with the instrument alignment mechanism,
applying force with orthopedic instruments, such that forces applied by the orthopedic instrument are dissipated across the surface area of the base.

18. A method for establishing an insertion pathway in a patient for surgical instruments comprising the steps of:
placing a base having a patient contact surface area onto the patient such that applied forces are dissipated across the surface area,
inserting an instrument alignment mechanism onto the base, and
locking the instrument alignment mechanism into a position establishing a repeatable trajectory for the insertion of surgical instruments.

19. The method of claim 18 wherein the working cannula includes a proximal end and a distal end and wherein the cannula is inserted through the patient's soft tissue and the distal end is anchored in bone to preserve the insertion pathway.

20. A medical device for dissipating force comprising:
means for contacting a patient surface and dissipating applied forces over the patient surface, and
means for aligning instruments selectively positionable in at least 2 degrees of freedom with respect to the means for contacting.

21. A medical device for dissipating force comprising:
a plurality of bases, each of the plurality of bases having a patient contacting surface, the patient contacting surface including a surface area adapted for placement on a patient's body such that applied forces are dissipated across the surface areas of the plurality of bases;
each of the plurality of bases having an instrument alignment mechanism positionable in at least 2 degrees of freedom thereon;
each of the instrument alignment mechanisms being operably connected to each of the other instrument alignment mechanisms at a juncture outside a perimeter of the plurality of bases.

22. A method for aligning surgical instruments comprising the steps of:
providing a base having a patient contact surface area,
providing an instrument alignment mechanism, and
providing instructions for using the base and instrument alignment mechanism
including:
placing the base on the patient surface,
positioning the instrument alignment mechanism on the base, and
locking the instrument alignment mechanism into position establishing a repeatable trajectory for the insertion of surgical instruments.

23. The method of claim 22 further including the step of positioning at least one orthopedic instrument to interface with the instrument alignment mechanism.

24. The method of claim 23 further including they step of applying force with orthopedic instruments, such that forces applied by the orthopedic instrument are dissipated across the surface area of the base.
